(19) United States
(12) Patent Application Publication
(10) Pub. No.: US 2017/0322220 A1
(43) Pub. Date: Nov. 9, 2017

(54) METHOD OF EXTRACTING ORGAN METABOLIC FUNCTIONS USING ORAL GLUCOSE TOLERANCE TESTS AND DEVICE THEREFOR

(71) Applicant: UNIVERSITY OF ULSAN FOUNDATION FOR INDUSTRY COOPERATION, Ulsan (KR)

(72) Inventor: Chae-Hun LEEM, Seoul (KR)

(73) Assignee: UNIVERSITY OF ULSAN FOUNDATION FOR INDUSTRY COOPERATION, Ulsan (KR)

(21) Appl. No.: 15/522,778

(22) PCT Filed: Aug. 25, 2015
(86) PCT No.: PCT/KR2015/008858

§ 371 Date: Apr. 28, 2017

(30) Foreign Application Priority Data
Oct. 31, 2014 (KR) 10-2014-0150329
Aug. 20, 2015 (KR) 10-2015-0117400

(51) Int. Cl. G01N 33/66; G01N 33/74; G06F 19/12

(52) U.S. Cl. CPC G01N 33/66; G06F 19/12; G01N 33/74; G01N 2333/62; G01N 2800/7071; G01N 2800/042

(57) ABSTRACT

A method of extracting organ metabolic functions using oral glucose tolerance tests (OGTTs) comprises: a step of measuring an amount of change over time in plasma glucose and plasma insulin at a regular time interval after a subject has consumed a certain quantity of a glucose solution; a step of extracting parameters associated with organ metabolism in a human body by applying the amount of change over time of the measured glucose and insulin to a human organ function model; and a step of diagnosing the metabolic function state of the subject using the extracted parameters.

### TECHNICAL FIELD

The present invention relates to a method of extracting organ metabolic functions using oral glucose tolerance tests and a device therefor, more particularly, a method of extracting organ metabolic functions using oral glucose tolerance tests using an oral glucose tolerance test capable of accurately diagnosing a metabolic function state of a subject or a patient and a device therefor.

### BACKGROUND ART

Oral glucose tolerance tests (OGTTs) are a test method for determining by measuring glucose levels after taking prescribed sugars and mainly used to diagnose diabetes mellitus (DM).

Insulin blood glucose data and changes induced by oral glucose tolerance tests (OGTTs) include information on intestinal absorption, glucose and insulin control in liver, pancreatic insulin secretion, glucose and insulin control in peripheral tissues.

Accordingly, an appropriate dynamic model may represent the above information from oral glucose tolerance tests (OGTTs).

However, diabetes has been diagnosed by using only blood glucose level after fasting for 12 hours and blood glucose level for 2 hours after glucose intake. The diagnosis was based on epidemiological data without any basis for the physiological mechanism to have a problem that it is difficult to accurately diagnose it based on mechanism.

The technology of the background of the present invention is disclosed in Korean Patent No. 10-0902282 (published on Jun. 10, 2009).

### DISCLOSURE

### Technical Problem

To solve the above problems, an object of the present invention is to provide a method of extracting organ metabolic functions using oral glucose tolerance tests using an oral glucose tolerance test capable of accurately diagnosing a metabolic function state of a subject or a patient, and a device therefor.

### Technical Solution

To achieve the object of the present invention, a method of extracting organ metabolic functions using oral glucose tolerance tests (OGTTs) according to an aspect of the present invention comprises ingesting a dose of glucose solution to a subject and measuring an amount of change over time in plasma glucose and plasma insulin at a regular time interval; extracting parameters related to organ metabolism in a human body by applying the change over time in plasma glucose and plasma insulin to a human organ function model; and diagnosing metabolic function state of the subject using extracted parameters.

The human organ function model may comprise a glucose compartment and an insulin compartment, in which the glucose compartment comprises a glucose compartment in a liver and a blood glucose compartment in plasma, and the insulin compartment comprises compartments of plasma insulin, liver insulin, peripheral insulin, liver receptor and peripheral receptor.

The extracted parameters may comprise at least one among gastrointestinal glucose absorption rate, insulin reduction rate, number of receptors in liver, number of receptors in peripheral tissues, glucose sensitivity for insulin secretion, glucose response to insulin secretion, maximum insulin production rate, maximum insulin-dependent glucose uptake rate in peripheral tissues, liver glucose maximum production rate, maximum ratio of insulin-dependent glucose produced from liver and glucose absorption rate to liver.

The step of diagnosing metabolic function state of the subject may determine the extracted parameter with a reference value whether the metabolic function of the subject is normal.

According to another aspect of the present invention, a device for extracting organ metabolic functions using oral glucose tolerance tests (OGTTs) comprises a measuring unit for measuring an amount of change over time in plasma glucose and plasma insulin at a regular time interval after ingesting a dose of glucose solution to a subject; a modeling unit for modeling a human organ function model comprising a glucose compartment and an insulin compartment; a parameter extracting unit for extracting parameters related to organ metabolism in a human body by applying the change over time in plasma glucose and plasma insulin to a human organ function model; and a diagnosis unit for diagnosing metabolic function state of the subject using extracted parameters.

### Advantageous Effects

As described above, according to the present invention, a metabolic function state of a subject or a patient can be diagnosed to provide the exact metabolic function and a basis for the disease, by extracting the glucose absorption function of the gastrointestinal tract, the hepatic glucose treatment function, the insulin secretion function of the pancreas relative to the blood glucose level, and the glucose metabolism function of the terminal tissues.

It is also possible to identify the metabolism function of each individual by using it, apply it to appropriate health management system, and establish a new diagnostic standard based on the physiological mechanistic basis rather than the conventional diabetes standard.

In addition, it can also be used in a variety of areas such as health check-ups of the clinic, wellness platform which is a present national concern, and other personal health care programs.

### DESCRIPTION OF DRAWINGS

FIG. 1 illustrates a diagram of a device for extracting metabolic functions using an oral glucose tolerance tests (OGTTs) according to an embodiment of the present invention.

FIG. 2a is a diagram for explaining a human organ function model based on a physiological system according to an embodiment of the present invention, and FIG. 2b is a diagram showing a portion to which Equations 6 to 20 are applied in the human organ function model shown in FIG. 2a.

FIG. 3 is graphs showing changes in glucose and insulin during the OGTTs.

FIG. 4 is a flow chart of a method of extracting metabolic function using oral glucose tolerance tests (OGTTs) according to an embodiment of the present invention.

### BEST MODE

Hereinafter, examples of the present invention will be described in detail with reference to the accompanying drawings. The terms used are terms selected in consideration of the functions in the examples, and the meaning of the terms may vary depending on the subject, the intention of the operator, or the precedent, and the like. Therefore, the meaning of the term used in the following examples is defined according to the definition when it is specifically defined in this specification, and unless otherwise defined, it should be interpreted in a sense generally recognized by those skilled in the art.

The device for extracting metabolic function using the oral glucose tolerance tests (OGTTs) according to an embodiment of the present invention extracts metabolic functions of the gastrointestinal tract, liver, pancreas and other tissues (muscle/fat) in the human body by using glucose and insulin concentration change data of the oral glucose tolerance tests (OGTTs) of the physiological model, which has been used for diabetes diagnosis conventionally.

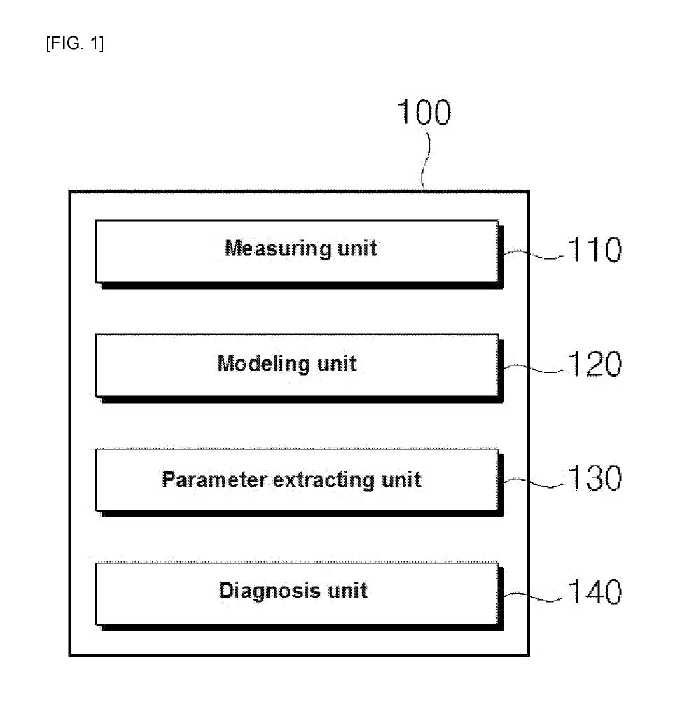

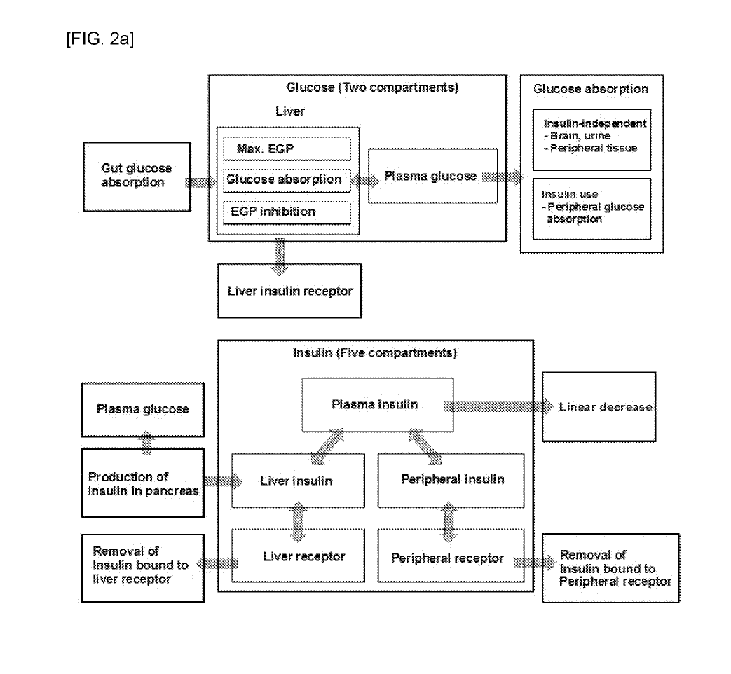

FIG. 1 shows a diagram of a device for extracting metabolic functions using an oral glucose tolerance tests (OGTTs) according to an embodiment of the present invention.

As shown in FIG. 1, a device 100 for extracting organ metabolic functions using oral glucose tolerance tests (OGTTs) according to an embodiment of the present invention comprises a measuring unit 110, a modeling unit 120, a parameter extracting unit 130 and a diagnosis unit 140.

Firstly, the measurement unit 110 measures an amount of change over time in glucose and insulin at a regular time interval after the subject ingests a dose of glucose solution.

The modeling unit 120 models a human organ function model based on a physiological system comprising a glucose compartment and an insulin compartment.

The parameter extracting unit 130 extracts the parameters related to the organ metabolism of the human body by applying the change over time in the measured glucose and insulin to the human organ function model.

The diagnosis unit 140 diagnoses the metabolic function state of the subject using the extracted parameters.

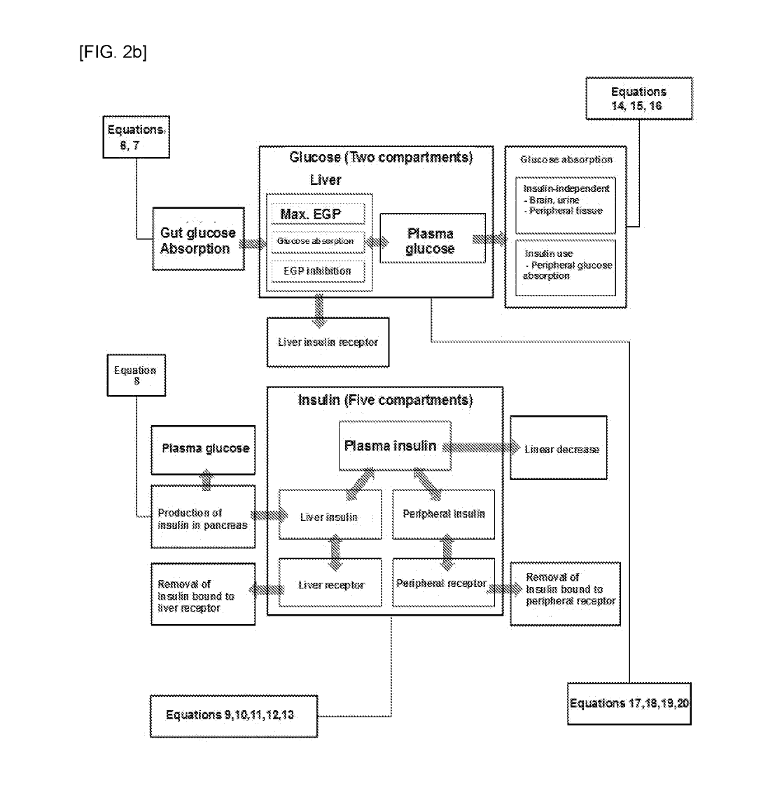

Hereinafter, a human organ function model based on a physiological system for extracting metabolic functions according to an embodiment of the present invention will be described with reference to FIGS. 2a and 2b.

FIG. 2a is a diagram for explaining a human organ function model based on a physiological system according to an embodiment of the present invention, and FIG. 2b is a diagram showing a portion to which Equations 6 to 20 are applied in the human organ function model shown in FIG. 2a.

Firstly, the subject ingests a certain amount of glucose solution for oral glucose tolerance tests (OGTTs), and at each time point (at least 30 minutes, 60 minutes, 90 minutes, 120 minutes, 180 minutes: up to 24 hours per minute), the changes in plasma glucose and insulin in plasma (additionally incretin: gastrointestinal hormone such as gastric inhibitory peptide, glucagon-like peptide-1, glucagon) are measured.

As shown in FIG. 2a, the human organ function model based on the physiological system according to an embodiment of the present invention is divided into glucose and insulin compartments. In addition, the glucose compartment consists of two compartments (G[0], G[1]) and the insulin compartment is composed of five compartments (I[0], I[1], I[2], I[3], I[4]).

The glucose model is divided into the plasma glucose compartment (G[0]) and the glucose compartment (G[1]) generated in the liver.

Firstly, glucose which is orally ingested and then absorbed by the gut is transferred to the liver. Secondly, hepatic glucose which is partially absorbed from the gut and partially generated from the liver is transferred to the plasma through the bloodstream.

Plasma glucose is consumed or excreted through brain, peripheral tissue, urine, organs where insulin is not necessary, or absorbed in peripheral tissues where insulin is necessary.

The insulin model is divided into five compartments: plasma insulin, liver insulin, peripheral insulin, liver receptor and peripheral receptors. The insulin model also uses an amount of insulin reduction in plasma insulin, liver receptor and peripheral receptor compartments. Insulin is produced in the pancreas in response to plasma glucose and is transferred to the liver which is bound to hepatic insulin receptors and removed.

In the peripheral space, plasma insulin is delivered to the interstitial space bound to insulin receptors on the peripheral tissues (mainly muscle and fat) and decreases linearly.

In order to model the human organ function model according to the embodiment of the present invention, initial values may be set as shown in the following Equations 1 to 5.

1. Setting Initial Value

V[0]=0.04505×mWeight (L) [Equation 1]

V[1]=0.00495×mWeight(L) [Equation 2]

V[2]=0.15×mWeight (L) [Equation 3]

BSA=0.007184×mWeight^{0.425}×mHeight^{0.725}(m²) [Equation 4]

CO=0.04×BSA×HR(L/min) [Equation 5]

In Equation 1, V[0] is systemic plasma volume and is calculated as a ratio of 0.04505 L/kg to body weight. In Equation 2, V[1] is liver plasma volume and is calculated as a ratio of 0.00495 L/kg to body weight. In Equation 3, V[2] is peripheral insulin volume and is calculated as a ratio of 0.15 L/kg to body weight. The body surface area (BSA) is calculated using the Du Bois formula as shown in Equation 4 and the cardiac output (CO, L/min) is calculated using heart rate (HR) and body surface area (BSA) shown in Equation 5.

Table 1 shows the units of the respective components shown in the Equations 1 to 5.

TABLE 1

| Name | Description | Unit |
|---|---|---|
| V[0] | Systemic plasma volume | L |
| V[1] | Liver plasma volume | L |
| V[2] | Peripheral insulin volume | L |
| BSA | Body surface area | m² |
| CO | Cardiac output | L/min |
| HR | Heart rate | bpm |
| mWeight | Body weight | kg |
| mHeight | Body height | cm |

2. Gut Glucose Absorption

$$\begin{matrix} G [ 4 ] = 75000.0 \times {e}^{( - F 0 \times time )} ( mg ) & [ Equation 6 ] \\ \frac{dGut}{dt} = F 0 \times ( G [ 4 ] - 75000.0 \times {e}^{( - F 0 \times 600.0 )} ) ( mg min ) & [ Equation 7 ]\end{matrix}$$

In Equation 6, G[4] represents the amount of glucose remaining in the gut, and Equation 7 represents the glucose absorption rate of the gut. In

Equation 7, ‘F0’ means the gut glucose absorption rate, and the total glucose amount is assumed to be 75 g. In this model, it is assumed that there is no glucose remaining after 600 minutes.

3. Pancreas Insulin Production

$$\frac{dIns}{dt} = \frac{F 6}{{( \frac{F 4}{{G}_{[ 0 ]}} )}^{F5} + 1} ( mU min ) \qquad [ Equation 8 ]$$

Equation 8 shows the rate of insulin secretion in the pancreas, ‘Ins’ means insulin production quantity, and F4 to F6 parameters control insulin production. F4 is the maximum insulin secretion and the half activation concentration of glucose, which means glucose sensitivity for insulin secretion. F5 is the “Hill coefficient”, glucose response to insulin secretion, and F6 is the maximum insulin production rate.

4. Insulin Compartments

$$\begin{matrix}\frac{dI [ 0 ]}{dt} = 0.3 \times \frac{CO}{V [ 1 ]} \times I [ 1 ] - 0.3 \times \frac{CO}{V [ 0 ]} \times I [ 0 ] + 0.04628 \times \frac{V [ 0 ]}{V [ 2 ]} \times I [ 2 ] - 0.04628 \times I [ 0 ] - 0.0371 \times I [ 0 ] & [ Equation 9 ] \\ \frac{dI [ 1 ]}{dt} = 0.3 \times \frac{CO}{V [ 0 ]} \times I [ 0 ] - 0.3 \times \frac{CO}{V [ 1 ]} \times I [ 1 ] + 0.290608 \times \frac{V [ 2 ]}{250.0 \times V [ 1 ]} \times ( F 2 - I [ 3 ] ) \times I [ 1 ] + 0.024588 \times I [ 3 ] + \frac{dIns}{dt} & [ Equation 10 ] \\ \frac{dI [ 2 ]}{dt} = 0.04628 \times I [ 0 ] - 0.04628 \times \frac{V [ 0 ]}{V [ 2 ]} \times I [ 2 ] - \frac{0.290608}{250} \times ( F 3 - I [ 4 ] ) \times I [ 2 ] + 0.024588 \times I [ 4 ] & [ Equation 11 ] \\ \frac{dI [ 3 ]}{dt} = 0.290608 \times \frac{v [ 2 ]}{250.0 \times V [ 1 ]} \times ( F 2 - I [ 3 ] ) \times I [ 1 ] - 0.024588 \times I [ 3 ] - F 1 \times I [ 3 ] & [ Equation 12 ] \\ \frac{dI [ 4 ]}{dt} = \frac{0.290608}{250.0} \times ( F 3 - I [ 4 ] ) \times I [ 2 ] - 0.024588 \times I [ 4 ] - F 1 \times I [ 4 ] & [ Equation 13 ]\end{matrix}$$

In Equation 9, I[0] is plasma insulin, I[1] in Equation 10 is liver insulin and I[2] in Equation 11 is peripheral insulin. In Equation 12, I[3] is liver receptor insulin, and I[4] in Equation 13 is peripheral receptor insulin. As described in the above, ‘CO’ means cardiac output, and the hepatic blood flow is calculated as 30% of cardiac output (CO) based on existing circulatory physiology knowledge.

Parameters of F1 to F3 are used in the Equations 9 to 13, ‘F1’, ‘F2’ and ‘F3’ represent an insulin degradation rate, a receptor number on liver and receptor number on peripheral tissue, respectively.

5. Brain and Urine Glucose Uptake

$$\begin{matrix} \frac{dBrain}{dt} = 60.0 ( mg \text{/} min ) & [ Equation 14 ] \\ \frac{dUrine}{dt} = \frac{1}{600} \times {( G [ 0 ] - 200 )}^{2} ( if G [ 0 ] > 200 ) ( mg \text{/} min ) & [ Equation 15 ] \\ \frac{dUrine}{dt} = 0 ( if G [ 0 ] \leq 200 ) ( mg \text{/} min ) & [ Equation 16 ]\end{matrix}$$

A brain glucose uptake was assumed to be constant over time and was set at 60 mg/min as shown in Equation 14. Namely, it is assumed that the brain consumes 60 mg of glucose per minute.

Equation 15 represents urine glucose uptake and urine glucose uptake is considered only when blood glucose (G[0]) is greater than 200 mg/dl as shown in Equation 15, and is not considered when blood glucose (G[0]) is less than 200 mg/dl.

6. Glucose Compartments

$$\begin{matrix}\frac{dG [ 0 ]}{dt} = 0.3 \times CO \times 10.0 \times ( G [ 1 ] - G [ 0 ] ) - ( \frac{dG [ 2 ]}{dt} + \frac{dBrain}{dt} + \frac{dUrine}{dt} ) & [ Equation 17 ] \\ \frac{dG [ 1 ]}{dt} = 0.3 \times CO \times 10.0 \times ( G [ 0 ] - G [ 1 ] ) + \frac{dGut}{dt} + \frac{dG [ 3 ]}{dt} & [ Equation 18 ] \\ \frac{dG [ 2 ]}{dt} = mWeight \times ( 1 + F 7 \times \frac{I [ 4 ]}{F 3} ) \times ( 1 - {e}^{( - 0.007 \times G [ 0 ] )} ) & [ Equation 19 ] \\ \frac{dG [ 3 ]}{dt} = mWeight \times F 8 - mWeight \times ( F 1 \; 0 + F 9 \times \frac{I [ 3 ]}{F 2} ) \times G [ 1 ] & [ Equation 20 ]\end{matrix}$$

The glucose compartment is basically composed of two compartments (G[0] and G[1]), G[0] in Equation 17 is plasma glucose, G[1] in Equation 18 is liver glucose. In this model, G[2] and G[3] were additionally considered. G[2] is the peripheral glucose consumption and G[3] is the liver glucose production.

As shown in Equation 19, G[2] is determined by F3 and F7, ‘F3’ is the number of peripheral tissue insulin receptor and ‘F7’ is the maximal insulin dependent glucose uptake rate in peripheral tissue.

As shown in Equation 20, G[3] is determined by F2, F8, F9 and F10, ‘F2’ is the liver insulin receptor number, ‘F8’ is the liver glucose maximum production rate, ‘F9’ is the maximal insulin dependent inhibition rate of glucose production from liver, and ‘F10’ is the glucose uptake rate into the liver.

Table 2 shows definitions and units of F0 to F10 shown in the above equations.

TABLE 2

| Name | Description | Unit |
|---|---|---|
| F0 | Gut glucose absorption rate | min⁻¹ |
| F1 | Insulin receptor bounded insulin degradation | min⁻¹ |
| | rate | |
| F2 | Liver insulin receptor number | mU/kg |
| F3 | Peripheral tissue insulin receptor number | mU/kg |
| F4 | Insulin secretion glucose sensitivity | mg/dl |
| F5 | Insulin secretion slope factor | unitless |
| F6 | Insulin maximum production rate | mU/min/kg |
| F7 | Peripheral insulin dependent glucose uptake | mg/min/kg |
| | rate | |
| F8 | Liver glucose maximum production rate | mg/min/kg |
| F9 | Liver insulin dependent glucose production | dl/min/kg |
| | inhibition rate | |
| F10 | Liver glucose uptake rate | dl/min/kg |

Table 3 shows definitions and units of G0 to G4, Gut, I0 to I4, and Ins shown in the above equations.

TABLE 3

| Name | Description | Unit |
|---|---|---|
| G[0] | Plasma glucose | mg/dl |
| G[1] | Liver glucose | mg/dl |
| G[2] | Peripheral glucose consumption | mg |
| G[3] | Liver glucose production | mg |
| G[4] | Remained glucose in gut | mg |
| Gut | Absorbed gut glucose | mg |
| I[0] | Plasma insulin | mU/kg |
| I[1] | Liver insulin | mU/kg |
| I[2] | Peripheral insulin | mU/kg |
| I[3] | Liver receptor insulin | mU/kg |
| I[4] | Peripheral receptor insulin | mU/kg |
| Ins | Insulin production quantity | mU/kg |

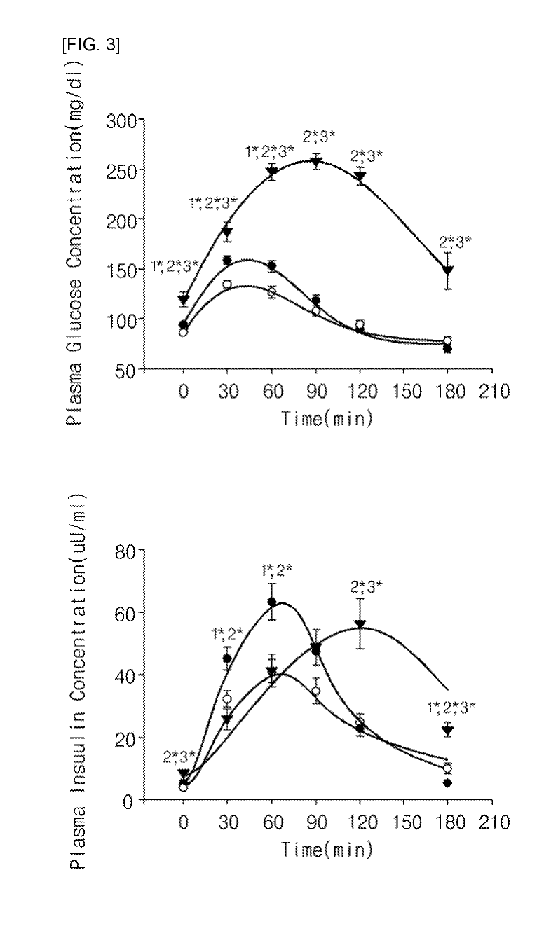

FIG. 3 is a graph showing changes in glucose and insulin during the OGTTs.

FIG. 3 shows that even when the same amount (75 g) of glucose is ingested by each group, it has different characteristics, and that the change amount in glucose and insulin is greater in male () than female (◯). Especially, insulin levels at 180 minutes were significantly lower in males than females.

In diabetic patients (q), the changes in glucose and insulin are different to each other. Basal plasma glucose was much higher in diabetic subjects and oral glucose load increased plasma glucose levels leading to hyperglycaemia. However, the increase in insulin was small and reached a peak value later than that in normal cases.

A solid line in FIG. 3 represents a line calibrated by the model of the present invention. For males, R² for glucose and insulin were 0.99 and 0.97, respectively, 0.97 and 0.94 for females and 0.96 and 0.86 for diabetics, respectively.

Table 4 shows the fitted parameters and F7/F3 represents the transfer rate of peripheral glucose to the insulin receptor (I[4]). F9/F2 represents the transfer rate of hepatic glucose to the insulin receptor (I[3]).

TABLE 4

| | Normal male | Normal female | Diabetic Subject |
|---|---|---|---|
| F0(×10⁻³ min⁻¹) | 7.56 ± 0.44^{1*} | 4.05 ± 0.21^{1*,3*} | 7.73 ± 1.19^{3*} |
| F1(×10⁻² min⁻¹) | 1.36 ± 0.11 | 1.21 ± 0.19 | 1.34 ± 0.06 |
| F2(mU/kg) | 259.5 ± 3.8 | 267.0 ± 4.1³ | 248.1 ± 5.3³ |
| F3(mU/kg) | 269.3 ± 3.0 | 254.9 ± 4.2 | 265.1 ± 6.4 |
| F4(mg/dL) | 134.4 ± 5.2^{1*,2*} | 116.9 ± 2.6^{1*,3*} | 173.8 ± 7.1^{2*,3*} |
| F5 | 10.25 ± 0.68 | 11.42 ± 0.74³ | 8.13 ± 1.64³ |
| F6(mU/min/kg) | 2.42 ± 0.20^{1*,2*} | 1.71 ± 0.16^{1*,3*} | 1.17 ± 0.11^{2*,3*} |
| F7(mg/min/kg) | 448.6 ± 56.4 | 380.6 ± 46.7 | 321.1 ± 51.4 |
| F8(mg/min/kg) | 3.87 ± 0.14² | 4.21 ± 0.20^{3*} | 3.28 ± 0.14^{2,3*} |
| F9(×10⁻¹ | 0.98 ± 0.24³ | 1.50 ± 0.33^{3*} | 0.10 ± 0.00^{2*,3*} |
| dL/min/kg) | | | |
| F10(×10⁻² | 1.71 ± 0.09^{3*} | 1.75 ± 0.10^{3*} | 1.00 ± 0.00^{2*,3*} |
| dL/min/kg) | | | |
| F7/F3(mg/min/mU) | 1.70 ± 0.22 | 1.45 ± 0.18 | 1.22 ± 0.21 |
| F9/F2(×10⁻⁴ | 3.71 ± 0.86 | 5.52 ± 1.16^{3*} | 0.40 ± 0.00^{2*,3*} |
| dL/min/mU) | | | |
| Basal | 1.82 ± 0.08¹ | 2.24 ± 0.18¹ | 1.95 ± 0.10 |
| EGP(mg/min/kg) | | | |
| γ² for glucose | 0.97 ± 0.004 | 0.96 ± 0.008 | 0.95 ± 0.013 |
| γ³ for insuulin | 0.95 ± 0.008 | 0.93 ± 0.020 | 0.83 ± 0.029 |

There were obvious gender differences in a number of calibrated parameters, including F0, F4, F6, and basal endogenous glucose production (EGP).

However, gender differences did not show in the diabetic group. Therefore, all data do not distinguish gender in the diabetic group.

There were big differences between parameters of normal male and diabetic patients (F4, F6, F8, F9, F10, F9/F2) and between parameters of normal female and diabetic patients (F0, F2, F4, F6, F8, F9, F9/F2). Furthermore, the difference between the normal case and the diabetic patient was remarkable in pancreas and liver.

The model according to embodiments of the present invention can account for many important physiological aspects of normal and diabetic patients. In particular, the model according to an embodiment of the present invention shows the difference between male and female according to absorption rate of glucose, endogenous glucose production (EGP), glucose sensitivity in pancreas, maximum insulin production capacity.

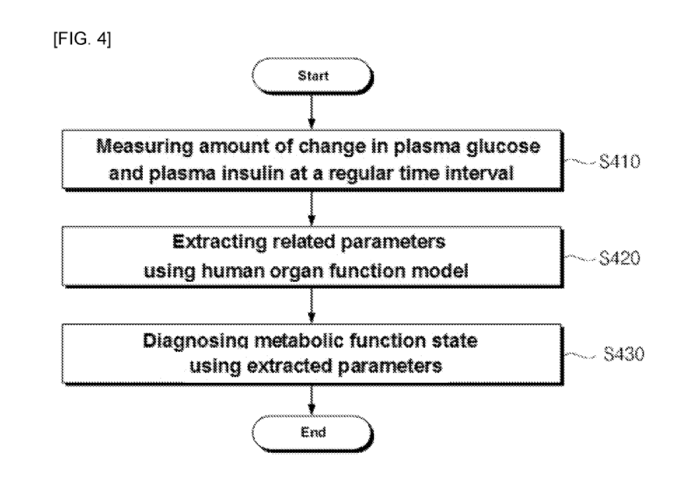

FIG. 4 is a flow chart of a method of extracting metabolic function using oral glucose tolerance tests (OGTTs) according to an embodiment of the present invention.

Firstly, the subjects ingest a dose of glucose solution for oral glucose tolerance tests (OGTTs) and a change in plasma insulin (I[0]) is measured at each time point (for example, every 30 minutes) (S410).

Next, the parameter extracting unit 120 applies a change amount of the measured plasma glucose (G[0]) and plasma insulin (I[0]) according to time to the human organ function model, thereby extracting parameters related to human organ metabolism (S420).

Namely, a change amount of change according to time in plasma glucose (G[0]) and plasma insulin (I[0]) is applied to Equations 6 to 20 to extract unknown parameters related to organ metabolism.

Here, unknown parameters include at least one among gut glucose absorption rate [F(0)], insulin reduction rate [F(1)], number of receptors in liver [F(2)], number of receptors in peripheral tissues [F(3)], maximum insulin secretion and the half activation concentration of glucose [(F4)], glucose sensitivity for insulin secretion [F(5)], glucose response to insulin secretion and maximum insulin production rate [F(6)], maximum insulin-dependent glucose uptake rate in peripheral tissues [F(7)], liver glucose maximum production rate [F(8)], maximum ratio of insulin-dependent glucose produced from liver [F(9)] and glucose absorption rate to liver [F(10)].

Next, the diagnosis unit 130 diagnoses the metabolism function state of the subject using the extracted parameters (S430).

The diagnosis unit 130 compares the extracted parameter with a reference value to determine if the metabolism function of the subject is normal, wherein the reference value may use parameter calibrated as shown in Table 4.

That is, even if the male and female have normal blood glucose levels and are not afflicted with diabetes, the extracted parameters are out of the range of the calibration parameters, comparing with parameters calibrated as shown in Table 4 and condition can be diagnosed for the corresponding items.

For example, when the liver glucose maximum production rate (F[8]) is out of the range shown in Table 4, treatment for the glucose production rate in the liver can proceed in advance even if they are not diabetic patients.

As described above, according to the embodiment of the present invention, metabolic function states can be extracted from each organ using a human organ function model based on physiological system.

That is, according to the embodiment of the present invention, it is possible to know the state of human body metabolic function of a subject or a patient to present an accurate metabolic function judgment and basis of disease, which previously has not been checked, by extracting the gut glucose absorption function, the hepatic glucose treatment function, the insulin secretion function of the pancreas relative to the blood sugar and the glucose metabolism function of the peripheral tissue.

It is also possible to identify the metabolism function of each individual by using it, apply it to appropriate health management system, and establish a new diagnostic standard based on the physiological mechanistic basis rather than the conventional diabetes standard.

In addition, it can also be used in a variety of areas such as health check-ups of the clinic, wellness platform which is a present national concern, and other personal health care programs.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A method of extracting organ metabolic functions using oral glucose tolerance tests (OGTTs) comprising:
ingesting a dose of glucose solution to a subject and measuring an amount of change over time in plasma glucose and plasma insulin at a regular time interval;
extracting parameters related to organ metabolism in a human body by applying the change over time in plasma glucose and plasma insulin to a human organ function model; and
diagnosing metabolic function state of the subject using extracted parameters.

2. The method of extracting organ metabolic functions using oral glucose tolerance tests (OGTTs) according to claim 1, wherein the human organ function model comprises a glucose compartment and an insulin compartment, wherein the glucose compartment comprises a glucose compartment in a liver and a blood glucose compartment in a plasma, and the insulin compartment comprises compartments of plasma insulin, liver insulin, peripheral insulin, liver receptor and peripheral receptor.

3. The method of extracting organ metabolic functions using oral glucose tolerance tests (OGTTs) according to claim 2, wherein the extracted parameters comprise at least one among gut glucose absorption rate, insulin reduction rate, number of receptors in liver, number of receptors in peripheral tissues, glucose sensitivity for insulin secretion, glucose response to insulin secretion, maximum insulin production rate, maximum insulin-dependent glucose uptake rate in peripheral tissues, liver glucose maximum production rate, maximum ratio of insulin-dependent glucose produced from liver and glucose absorption rate to liver.

4. The method of extracting organ metabolic functions using oral glucose tolerance tests (OGTTs) according to claim 3, wherein a step of diagnosing metabolic function state of the subject comprises determining the extracted parameter with a reference value whether the metabolic function of the subject is normal.

5. A device for extracting organ metabolic functions using oral glucose tolerance tests (OGTTs) comprising:
a measuring unit for measuring an amount of change over time in plasma glucose and plasma insulin at a regular time interval after ingesting a dose of glucose solution to a subject;
a modeling unit for modeling a human organ function model comprising a glucose compartment and an insulin compartment;
a parameter extracting unit for extracting parameters related to organ metabolism in a human body by applying the change over time in plasma glucose and plasma insulin to a human organ function model; and
a diagnosis unit for diagnosing metabolic function state of the subject using extracted parameters.

6. The device for extracting organ metabolic functions using oral glucose tolerance tests (OGTTs) according to claim 5, wherein the human organ function model comprises a glucose compartment and an insulin compartment, wherein the glucose compartment comprises a glucose compartment in a liver and a blood glucose compartment in a plasma, and the insulin compartment comprises compartments of plasma insulin, liver insulin, peripheral insulin, liver receptor and peripheral receptor.

7. The device for extracting organ metabolic functions using oral glucose tolerance tests (OGTTs) according to claim 6, wherein the extracted parameters comprise at least one among gastrointestinal glucose absorption rate, insulin reduction rate, number of receptors in liver, number of receptors in peripheral tissues, glucose sensitivity for insulin secretion, glucose response to insulin secretion, maximum insulin production rate, maximum insulin-dependent glucose uptake rate in peripheral tissues, liver glucose maximum production rate, maximum ratio of insulin-dependent glucose produced from liver and glucose absorption rate to liver.

8. The device for extracting organ metabolic functions using oral glucose tolerance tests (OGTTs) according to claim 7, wherein the diagnosis unit determines the extracted parameter with a reference value whether the metabolic function of the subject is normal.
